(19) United States
(12) Patent Application Publication
(10) Pub. No.: US 2007/0225693 A1
(43) Pub. Date: Sep. 27, 2007

(54) Treatment and diagnostic systems for the eye

(76) Inventors: Dirk Muehlhoff, Jena (DE); Martin Wiechmann, Jena (DE); Mark Bischoff, Bad Berka (DE); Karsten Festag, Jena (DE)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/716,994

(22) Filed: Mar. 12, 2007

(30) Foreign Application Priority Data
Mar. 10, 2006 (DE) 10 2006 011 623.2
Nov. 10, 2006 (DE) 10 2006 053 580.4

(51) Int. Cl. A61B 18/18

(52) U.S. Cl. 606/10; 606/2

(57) ABSTRACT

The invention refers to a system to treat and/or diagnose a patient's eye. The system in this invention comprises several devices for the treatment and/or diagnosis of the eye as well as means for the logical linking of the devices with each other, such as systems for the spatial positioning of the devices relative to each other, configurations for the positioning of the patient and the eye to be treated relative to the respective devices, systems for the controlled supply of the devices with power and auxiliary power, and/or notification tools to transmit information or control commands between the above-mentioned devices, units, configurations and systems, and to put out information.

### RELATED APPLICATIONS

The present application claims the benefit of priority to German Patent Application Nos. 10 2006 011 623.2 filed on Mar. 10, 2006 and 10 2006 053 580.4 filed on Nov. 10, 2006. Said applications are incorporated by reference herein.

### FIELD OF THE INVENTION

This invention involves a diagnostic treatment system for a patient's eye by direct or indirect alignment of diagnostic and treatment systems.

### BACKGROUND OF THE INVENTION

It is a known fact that single devices, whose design is adapted to their respective indication are used to treat or diagnose the eye. Thus, if for example a vision correction is to be made using laser surgery on the cornea, devices will be used to diagnose the eye, such as slit lamps, optical devices for three-dimensional measuring of the cornea, devices for optical coherence tomography (OCT) or similar devices.

On the basis of the results achieved by using one or more of these devices, the following will lay out the treatment methods as well as the selection and availability of the treatment devices.

During treatment and prior to the procedure to correct vision, a flap-shaped cutout is created on the surface of the cornea using a so-called “flap blade” laser device, which is also known as a laser keratome or its mechanical counterpart, a microkeratome. The thickness of this flap is much smaller than the cornea. In order to create such a flap most comfortably and precisely, laser keratomes are used which create a treatment laser beam with pulse widths smaller than 10⁻¹² s. This allows the creation of locally contained breakthroughs in the cornea with an expansion of only very few micrometers.

These are so-called photo disruptions. By precisely aligning a number of these breakthroughs, the desired flap can be created and made to “hinge” properly.

Directly following the creation of this flap it is folded up and away from the inner part of the cornea in order to correct the vision and the appropriate amount of tissue is removed. This (photo-) ablation of tissue is performed using energy by means of a treatment laser beam, such as for example, an Excimer laser. These devices used to ablate organic tissue will be called ablation lasers in this document.

Following the treatment procedure, diagnostic devices are used to evaluate the results of the treatment and induce follow-up treatment if necessary.

This shows that the sequence of device use for the diagnosis or therapy treatment of the eye must be carefully and logistically evaluated. This specifically includes patient-related identification, diagnosis and/or therapy data as well as treatment-specific configuration or protocol data and control signals, so that the treating physician or the user can effectively set up the device matching the treatment at hand. Furthermore, the patient must be repositioned every time a device is used and the patient's eye must be adjusted accordingly in every instant.

This coordination or matching of the devices with the respective necessary treatment and diagnosis conditions for each patient is made more difficult at this point because the devices in question are often set up in different rooms or, if it is possible to have them in the same room, there is often too much distance between them.

Thus, it is necessary to record the individual data of each patient, to generate treatment data from this data, to enter this data into the device to be used, and to display and evaluate the treatment and diagnostic data. Besides entering the treatment data further patient data, such as identification data, must be entered.

Due to the use of logistically separated state of the art devices, the treatment becomes time-intensive and they hold numerous sources for errors based on their repeated need for data entry and manual data transmission between devices.

Furthermore, it is known that patients have to be transported on a stretcher or a chair from one device to the next. This alone, however, does not guarantee a precise positioning of the patient for the treatment and/or diagnosis on the respective device.

As the configurational dimensions of the devices are not synchronized with one another, it is often necessary to transport the patient, not only on one level, but to also change their height position between devices according to their individual design.

### SUMMARY OF THE INVENTION

Assuming state of the art technology the object of this invention lies in the creation of a system of the type described in the beginning of this document, which will ensure a timely and swift sequence of events regarding the consecutive use of the devices needed to reduce the sources for errors and to increase the protection of the patient from possible injuries.

This object is resolved with a system to treat or diagnose a patient's eye which comprises several devices to diagnose and treat the eye as well as measures to logically link the devices with each other, namely

devices to position the devices in relation to one another,

configurations to position the patient and the eye to be treated relative to the respective devices,

systems for the controlled supply of the devices with power or auxiliary power, and/or

notification tools to transmit information or control commands between the above-mentioned devices, instruments, configurations or systems and for the output of information.

In one design of the system in this patent several treatment and/or diagnostic devices are present, and they are utilized consecutively in a synchronized time frame. This system would also include notification/message tools which would transmit and display information—preferably to the device that is next in line—regarding whether the patient is positioned correctly for the subsequent device coming from the previous station/device for the intended treatment or diagnostics.

Furthermore, these notification tools can be used to transmit further information or control commands and thus initiate actions in the subsequent device.

These notification tools can comprise mechanical, acoustic, electronic, and/or opto-electronic configurations to capture, process, transmit, and output data related to the patient, the orientation of the patient's eye, and/or the treatment or diagnosis. For the capturing portion there can be configurations for manual data entry and/or for transmitting data from one or more of the devices in a data processing system or in data storage. The system can be equipped with at least one opto-electronic display for the output of the data.

This can include the following configurations:

for the capture, creation, and processing of patient-related identification, diagnosis and/or therapy data and for their transmission between devices,

for the capture and processing of treatment-related configuration data, protocol data, and/or control commands and their transmission between devices

for the capture, creation, processing, transmission, and output of data regarding

the safety of the patient when positioning the patient's eye relative to the respective device to be utilized.

For the treatment of the eye an embodiment of this system comprises a laser keratome as a preceding device to create the flap cut in the surface of the cornea and a device with an Excimer laser as the subsequent ablation laser device to ablate the tissue from the cornea.

Alternatively, a second embodiment may include a microkeratome as a preceding device to create the flap cut in the surface of the cornea of the eye and an ablation laser device as the subsequent device to ablate the tissue in the cornea.

Besides these devices for treatment the system can also comprise devices for the diagnosis of the eye such as slit lamps, optical systems for three-dimensional measuring, and for configurational control as per Scheimpflug, or systems for the optical coherence tomography (OCT), for the topography, or for waveform diagnostics or pachymetry. With these devices the information required to determine the treatment steps can be obtained.

Also, the information can be processed with these devices and certain software applications can generate data that can be used to control the laser keratome and/or the ablation laser device.

If these devices are included in the system of this invention and if they are linked via the listed notification/messaging tools in the way described above the treatment or diagnosis can take place much faster, as compared to the state of the art now, and the sources for treatment errors due to faulty data transmission can be reduced.

In order to increase the patient's safety even more, another aspect of the system includes a patient positioning system to position the patient relative to the devices as well as tools to guide and change positions of the patient positioning system, where

the eye to be treated is positioned in an initial preferred position in the treatment area of the preceding device in order to create a flap cut in the surface of the cornea,

the eye to be treated is positioned in a second preferred position in the treatment area of the subsequent device in order to ablate the tissue from the cornea,

the eye to be treated is positioned in a third preferred position in the measurement and monitoring area of a preceding or subsequent device in order to diagnose the eye, and where

systems that detect positions and/or lock patient positioning systems in the preferred position are intended as part of the notification tools.

The movement path from a preferred first position to the subsequent position can run randomly, but the change in position is preferred to be on a circular path where the preferred positions are located on this circular path, or the movement will take place on a straight path where the positions would be located on this line.

This invention intends that at least one enter and one exit position are planned for the patient besides the above-mentioned positions at which the patient positioning system can be stopped to either let the patient enter at the beginning of the treatment/diagnosis or exit at the end.

The means of the change in position of the patient positioning system may include at least one positioning drive connected to a positioning control. This positioning control may issue positioning commands to the positioning drive and there should may be devices to control and confirm preset positions.

The positioning may could consist of an input module to manually issue the positioning commands, such as a joystick, or with a control to automatically generate positioning commands based on detected distance or force measurement values. In order to measure distance measurement values an incremental or absolute measuring positioning measurement system or an optical positioning measurement system with a camera module connected to the positioning control can be used.

The invention also contemplates that the positioning control be connected to a device that controls the patient's and the patient's eye position fully automatically. This may be equipped with a navigation supported drive control which is fitted with elements to detect the eye's position and to guide the eye position's path. These may be connected to the positioning drives via a control module.

This drive control can also contain sensors to detect obstacles which would also have to be connected to the positioning drives via a control module. This allows avoiding obstacles, which may be especially useful regarding the different device configurations.

If a piece of contact glass on top of the eye is used for treatment, which is penetrated by the laser beam, a special headrest may be used to ensure the patient's safety. The contact glass can be mobile and there may be a safety mechanism containing a force readings recorder to move the headrest and the contact glass apart if the glass applies too much force on the eye. This protects the patient and avoid crushing injuries to the patient's eye.

The safety mechanism only moves the contact glass and the headrest apart, if a certain limit value is exceeded and it adjusts the relative positions of the contact glass and headrest if this limit value is not reached.

The system has a basic body equipped to hold the devices belonging to the system and brackets to attach the devices. Here every device in the system may be assigned one fixed position which would contribute to the accuracy in positioning the patient.

Furthermore the invention contemplates that the basic body is fitted with a base for forced guidance of the movement when changing positions of the patient positioning system. This forced guidance can be realized by guiding paths such as rails that may be inside the base. These can also be designed as grooves pivoting around a center point in a circular pattern. Here it may be especially advantageous if the rotation axis does not protrude in the center but toward the foot end of the positioning system.

The notification tools can include connections to wireless or wired transmission of data such as a peer-to-peer network, preferably as a server-based network, or a bus, star or ring network. If a wired transmission is planned, at least part of the required cable length may be stored inside the basic body.

In order to supply power and/or auxiliary power to the system the devices in the system may be connected to a central power supply and this central power supply may have at least one emergency backup power unit.

It has been shown that it has a comforting and calming effect on the patient, if the immediate vicinity of the head in its treatment position is illuminated with a comfortable color shade (such as light blue). Hence, the housing of the treatment laser can be at least partially transparent and thus “self-illuminated”.

### BRIEF DESCRIPTION OF THE DRAWINGS

FIG. 1 is a schematic elevational view of a Treatment/Diagnostic System for the Eye in accordance with the present invention;

FIG. 2 is a schematic plan view of a Treatment/Diagnostic System for the Eye in accordance with the present invention; and

FIG. 3 is a schematic plan view of a Treatment/Diagnostic System for the Eye in accordance with the present invention.

### DETAILED DESCRIPTION

This invention is explained in more detail in the following.

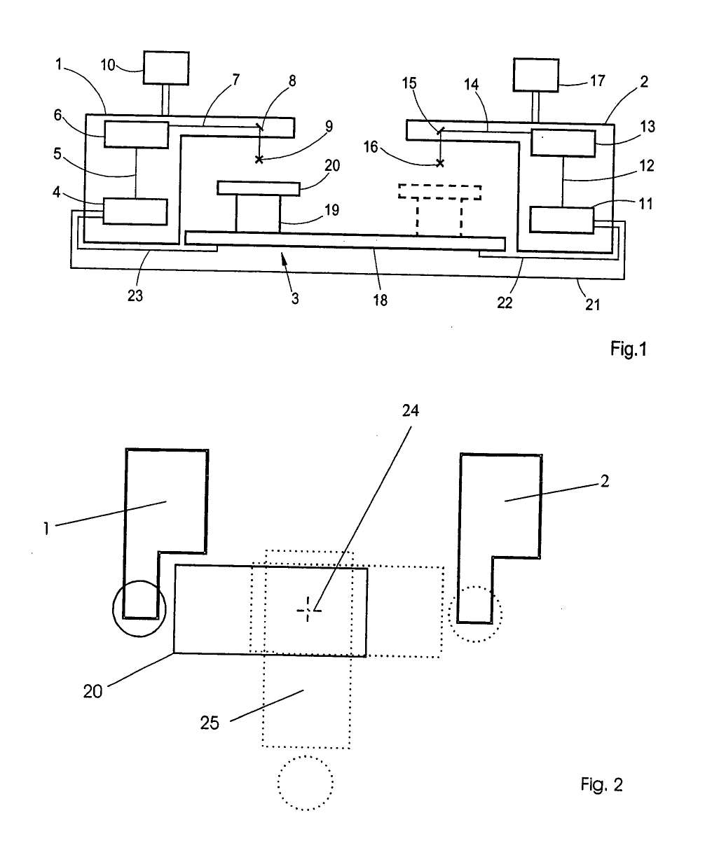

An associated drawing (FIG. 1) shows an example of the principle of the system in this invention for the treatment and diagnosis of the eye. The system comprises several treatment and diagnostic devices which will be used subsequently. Here, the preceding device is following by the subsequent one and so forth.

In order to elaborate on this principle, an example (FIG. 1) includes a laser keratome 1, an ablation laser device 2, and a patient positioning system 3, which is used by both devices.

The laser keratome 1 allows the use of Femto second laser pulses to perform state of the art cutting of the cornea and thus creating a “flap”. It includes a control unit 4, which triggers a laser source 6, via a control line 5. The laser source, 6, emits a laser beam 7, via a scanner 8 and an optical system (not shown) which is pointed toward an initial treatment area 9. The treatment and patient related data required for the treatment can be entered via an input device with a graphical user interface 10.

The ablation laser device 2 allows the ablation of corneal tissue from under the folded up flap by using UV laser beams via the known state of the art photo ablation. It includes a control unit 11, which controls, among other things, a laser source 13, via a control line, 12. The laser source 13, emits a UV laser beam 14, via a scanner 15, and an optical system (not shown) which is pointed toward an initial treatment area 16. The treatment and patient related data required for the treatment can be entered via an input device with a graphical user interface, 17.

The patient positioning system 3, includes an immobile base 18, a movement element 19, and a resting surface 20. The resting surface 20, can be repositioned in all three spatial dimensions (compared to the movement element 19) and thus coupled with motorized drives (not shown).

For the manual triggering and control of the movement a joystick is planned (not shown). An alternative to this joystick is that the control unit 4 of the laser keratome 1, and/or the control unit 11 of the ablation laser device 2, generate commands to trigger and control the movement and transmit these signals to the motorized drives.

The fact that the movement element 19, is mobile compared to the stationary base 18, is characteristic for this special configuration of the patient positioning system 3. Also, that at least two defined preferred positions are planned which communicate with the treatment areas 9 and 16 in a way that the positioning of the resting surface, 20, is the same relative to the treatment area 9 in an initial preferred position that is the same as the positioning of the resting surface 20, to the second treatment area 20 in the second preferred position.

In this context the system described in this invention comprises notification tools, which automatically provide information regarding whether the patient's eye that is to be treated has been positioned properly in the treatment area 9 of the laser keratome 1 (first position). They also automatically provide information as to whether the patient's eye to be treated has been moved properly from the laser keratome 1 via the movement element 19 to the ablation laser device 2 and whether it is ready for the subsequent treatment in the treatment area 16 of the ablation laser device 2 (second position).

In order to ensure this spatial relation, the movement of the movement element 19 and the resting surface 20 relative to the base 18 can either be restricted by mechanical stops; or the movement of the movement element 19 and the resting surface 20 relative to the base 18 can be controlled via units for positioning detection and positioning controls. These devices can be programmed to detect the respective desired preferred position and to stop the movement of the movement element 19 and the resting surface 20 once this position is reached.

The defined and fixed positions ensure that the patient is situated safely and reproducibly by the laser keratome 1 as well as the ablation laser device 2 regarding treatment. This also ensures that the patient will be moved from the first preferred position to the second preferred position without complications via the movement element 19 and that there will be no need for significant manual adjustments of more than 100 mm, such as moving the resting surface 20 relative to the movement element 19.

The movement of the movement element 19 relative to the base 18 can take place in different ways.

A first variant allows the movement element 19 to be perpendicular to the base 18 and it has the capability to rotate, so that the movement element 19 can move on a circular track with the resting surface 20 from one position to the next. This positions the resting surface 20 and the head, or rather the eye of the patient according to the treatment sequence: first in treatment area 9 of the laser keratome 1 and after the rotation in treatment area 16 of the ablation laser device 2. In other words: the two positions are positions on the circular track, which is traveled by the movement element 19 including the resting surface 20 in a rotating manner.

In an alternative example, the movement element 19 is stationary and the resting surface 20 travels on a rotating axis 24 as defined by the movement element 19.

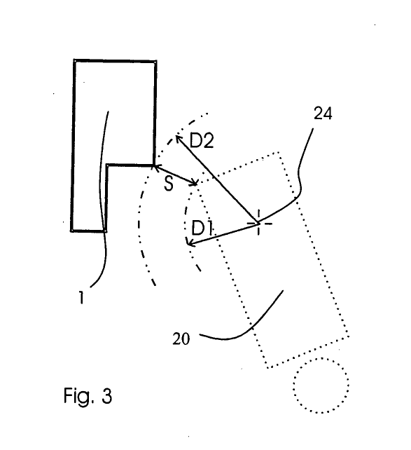

An example of this type is shown in FIG. 2 (top view). Here, the rotating axis 24 is not shown symmetrical to the resting surface 20, but it is moved to the foot end of the resting surface 20. This solution has advantages: for example, the foot end of the resting surface 20 will not collide/interfere with housing parts of the treatment lasers 1 and 2 even if it is turned by more than 90°. Furthermore, the alternative position 25 offers a comfortable entry and exit opportunity for the patient. FIG. 3 depicts the spatial conditions in more detail. The distance D1 between the rotating axis 24 and a corner of the foot end of the resting surface 20 must be one safety distance S smaller than the distance D2 of the rotating axis 24 from its nearest housing contour of the respective treatment laser 1 (or 2) to avoid collisions.

Alternative to its rotating movement, in a second variant, the movement element 19 can be moved from one preferred position to the other by means of translation. For this, the movement element 19 is moved on tracks or similar guiding devices which—in this case—are part of the base 18. The two preferred positions are then also positions on the track, which describes the movement element 19 including the resting surface 20 within the translation.

This invention also includes designs where the movement element 10 is equipped with a separate drive and moves from one preferred position to the next without running on tracks. Here, the detection of the preferred positions can take place via sensors which are suited to be integrated into marks in the base 18 or on the laser keratome 1 and on the ablation device 2 and can be precisely detected.

In both examples base 18 can be designed so that it extends from the basic area of laser keratome 1 on one side and to the basic area of the ablation laser device 2 on the other side. Furthermore, the base 18 can be designed to store extra cable length which may be used to transmit data between the laser keratome 1, the ablation laser device 2, and the patient positioning system 3. This base may be equipped with cable entry and exit areas.

In another design in the framework of this invention the base 18 is part of a base body (not shown) which the laser keratome 1 and the ablation laser device 2 are placed and mounted on with their bases. Here, it is conceivable applying positioning markers for the laser keratome 1 or the ablation laser device 2 or attachments for these devices on the base body.

Furthermore, base 18 and also the base body may be accessible by personnel/patient. The outer surrounding of the base 18 or the base body is then either designed vertically as a step, where the step height should not exceed 18 cm, or it may be fashioned at an angle as a ramp to minimize the risk of tripping when entered.

The surface of the base 18 or the base body should comply with the requirements of flooring in operating rooms in order to control the danger of slipping and to allow for efficient cleaning.

Independent of the above-described possibilities of triggering and controlling the movement of the movement element 19 the movement can be triggered or controlled using other state of the art methods, either manually or motor-driven. The manual option has the advantage that the person controlling the movement can directly observe to avoid collisions.

On the other hand, a motor-driven movement has the advantage that there is no effort on the part of the operator. Furthermore, a movement of this kind can also be put together using multiple partial movements. It is especially possible to design the movement sequences so that a position is left behind by the resting surface 20 being lowered first. Only then, the movement element 19 will move together with the resting surface 20 and the patient to the device that is intended to be next in the sequence of the treatment. After that, the resting surface is raised back up until the second position is reached. This movement on a U-shaped track will effectively prevent parts of the laser keratome 1 or the ablation laser device 2, which protrude into the treatment areas 9 or 16, from injuring the patient.

Furthermore, this provides the opportunity to arrange the treatment area 9 of the laser keratome 1 and the treatment area 16 of the ablation laser device 2 at different heights above the area of operation or the common base body. In this case, it is merely be necessary to indicate different travel paths for the height adjustment of the resting surface 20 relative to the two devices.

Contrary to this, it is of course an advantage if the devices have approximately the same construction so that the treatment areas 9 and 16 are at about the same height above the common area of operation. This will ensure that the height of the resting surface 20 with the patient will not have to be modified. Besides saving time, this also increases the safety of the patient.

To improve the patient's comfort the housings of one or both treatment lasers 1, 2 in the vicinity of the treatment areas 9 and 16 will have an indirect illumination, such as translucent housing models, which can generate colored light which is gentle on the patient's eyes.

Furthermore, the system has specific safety functions to prevent injuries to the patient's eye, especially crushing.

For this, the patient positioning system 3 is equipped with a position detector (not shown). This position detector comprises an incremental or absolute position measuring system to capture distance measuring values or an optical position measuring system with a camera module.

The positioning control is connected to a device for the completely automatic positioning of the patient or the patient's eye, which in turn comprises a navigation supported drive control and which is equipped with elements to capture the position of the eye and to trace its position. It also has sensors to detect obstacles and a control module which is connected to these sensors and the positioning drives to avoid obstacles.

This provides continuous information regarding the location of the patient positioning system 3, especially whether it is in one of the preferred positions, or, for example, if the patient has left one treatment position and is on his way to the next, or if he is still located between the positions. The accuracy of the position detection is at least approx. 100 mm, the ideal case would be merely 100 μm. Depending on the position and further signals, which the positioning system receives from one or both of the devices, certain movements of the patient positioning system are blocked in order to avoid collisions during the movement.

For example, all undesired movements of the resting surface 20 and the movement element 19 can be blocked that way if a contact glass has been placed on the patient's eye in the treatment area 9 of the laser keratome 1 which the treatment laser beam of the laser keratome 1 will penetrate if the laser is pointed at the patient's eye, so that crushing injuries can be prevented.

In a specifically advantageous and simple model the position detection is equipped with two switches which activate an initial partial quantity of interlock functions in the first preferred position and then a second partial quantity of interlock functions at the second preferred position. Here, the first partial quantity of interlock functions is controlled by the laser keratome 1 and the second partial quantity of interlock functions is controlled by the ablation laser device 2.

In order to ensure a smooth operation during treatment or diagnosis a data connection 21 can be established between the laser keratome 1 and the ablation laser device 2. This data connection 21 can be wired or wireless and does not have to be a direct connection between the two devices, but the transmission can take place via an inserted data processing devices or data storage devices. The data connection 21 is intended to scale patient-related data between the devices, to synchronize the controls of the devices regarding treatment parameters, and/or to transmit diagnostic parameters that are to be included in the treatment procedure.

Thus, a patient-related data set can contain the following data:

patient name and identification (birth date, file no., etc.)

identification of the eye

type of ametropia

treatment-related correction target values

diameter of the optical zone to be treated

flap diameter, flap thickness, cut guidance parameters of the flap edge

date of procedure/treatment.

First, this data is entered into a centrally located input mask or via the user interfaces 10, 17 on the devices. The data connection now transmits this data completely or partially to the other device so that they will be available for the subsequent treatment step. This will help avoid erroneous assignments of treatment data to patient data.

If the data is changed after it has been entered and transmitted to one or both devices, the data connection will be used to transmit these changes and to ensure that the data set is synchronized between the two devices at all times and that it is generally identical.

As already depicted, the data connection can be used to synchronize the control actions between the laser keratome 1 and the ablation laser device 2 so that user actions performed on one device during treatment will trigger actions on the other device. To illustrate this, the following will describe a possible treatment sequence:

In the beginning of the treatment procedure the patient's complete data is available on both devices. By means of the graphic user interface 10 on the laser keratome 1 a patient data set is selected from a database which is assigned to the patient to be treated. The treatment data for the flap and the subsequent ablation are displayed for the user. He can in turn check all entries at this point and change them before the treatment procedure starts. If values are changed at this time, which might affect the subsequent treatment with the ablation laser device 2, these changes are transmitted to the ablation laser device 2 via the data connection and will be available for the ablation following the creation of the flap.

However, before the flap cut is performed using the laser keratome 1 its control unit 11 transmits a command to the ablation laser device 2. In turn, the ablation laser device 2 performs a self-test and calibrates the laser source according to the treatment parameters. Only after the ablation laser device 2 has transmitted its readiness to perform a subsequent ablation to the laser keratome 1 via the data connection, the control unit 11 of laser keratome 1 will issue a release for the cutting procedure.

This measure ensures that a patient can be treated further without delay using the ablation laser device 2 after the flap cut has been performed. This will specifically prevent an unnecessary drying out of the cornea. Furthermore, the risk of an impossible further treatment of a patient due to a failure of the ablation laser device is significantly reduced.

After the release has been issued, and all data and parameters have been checked and confirmed by the user, the flap cut is performed on the eye to be treated using the laser keratome 1. After the cutting procedure is completed the patient positioning system 3, with the patient is moved from one position, where the patient's eye is still located in the treatment area of the laser keratome 1, to the other position, so that the eye is now positioned in the treatment area of the ablation laser device 2. Data connections 22 and 23 will trigger the patient positioning system 3.

At the same time, data from the control of the laser keratome 1 will be transmitted to the ablation laser device 2 which contains the patient information of the patient who was just treated with a flap cut. Subsequently, a window showing the patient data, already taking into consideration possible data regarding changes to the laser keratome 1 as well as the flap depth selected on the laser keratome 1, will be displayed on the graphic user interface 17 of the ablation laser device 2. The user must now simply confirm this data or adjust it if necessary and the ablation can begin.

After the ablation is completed the patient is either moved to a third position with the patient positioning system 3, which can be an entry or exit position of the patient and he is asked to get up, or he is returned to the first position to repeat the treatment procedure on the second eye which has not yet been treated. If the latter is the case the treatment data for the second eye will automatically be displayed on the graphic user interface 17 of laser keratome 1.

The system described in this example, which includes of the laser keratome 1, the ablation laser device 2, and the patient positioning system 3, can be supplemented with further devices, especially diagnostic devices to capture data required for the treatment with the above-mentioned treatment devices or to check the changes effected using these treatment devices. These can be diagnostic devices such as slit lamps, optical systems for 3D measuring and for geometrical control as per Scheimpflug, or systems for optical coherent tomography (OCT), for topography, for wave front diagnostics or, to measure the thickness following the pachymetry principle.

The invention further includes models of the system which would include a data transmission device such as a modem. Furthermore, computers can be used for the planning, control, and/or storage of treatment process data fashioned as planning and diagnostics units.

As previously described, the connections used to transmit the data between the devices, the patient positioning system, the data input and output units as well as the data processing systems, can be wired or wireless like a peer to peer network. The network and the data transmission protocols should be set up to keep the possibility of data loss and transmission errors to a minimum, while the use of a CAN bus should be preferred due to its excellent data transmission security.

1-26. (canceled)

27. A system for the treatment and/or diagnosis of a patient's eye, comprising:
a plurality of clinical devices for the treatment and diagnosis of the eye;
means for logistical linking of the clinical devices with each other, including
spatial positioning devices to spatially position the clinical devices relative to one another
a patient positioner to position the patient and the eye to be treated relative to the clinical devices;
control systems to control power and auxiliary energy supply to the devices and/or notification tools to transmit information or control commands between the clinical devices, spatial positioning devices, patient positioner and control systems, and to output information.

28. The system as claimed in claim 27, in which the clinical devices, include a preceding device that is used prior to a subsequent device in a time sequence, and
further in which the notification tools automatically transmit and output information regarding whether the patient has been positioned properly at a subsequent device after arriving from a preceding device.

29. The system as claimed in claim 27, wherein the notification tool includes mechanical, electronic, and/or opto-electronic interfaces to capture, process, transmit and output data related to the patient, the positioning of the patient's eye and/or the treatment or diagnosis procedure.

30. The system as claimed in claim 29, wherein patient-related identification, diagnostic, and/or therapy data is captured, processed and transmitted between the clinical devices.

31. The system as claimed in claim 29, wherein treatment-related configuration data, protocol data, and/or control signals are captured and processed and transmitted between the clinical devices.

32. The system as claimed in claim 29, wherein data regarding safety when positioning the patient's eye is captured, processed, transmitted, and output relative to the clinical devices.

33. The system as claimed in claim 27, wherein the notification tools further comprise at least one interface for manual data input and/or transfer of data from one or more devices to a data processing unit or into data storage, and at least one opto-electronic display unit.

34. The system as claimed in claim 27, wherein one of the clinical devices comprises a Femto second laser device that is used to create a flap cut in the surface of the cornea prior, and
another of the clinical devices comprises an Excimer laser that is used to ablate the cornea.

35. The system as claimed in claim 27, wherein one of the clinical devices comprises a laser keratome that is used to create a flap cut in the surface of a cornea, and another of the clinical devices comprises a stationary laser working in a UV range that is used for the ablation of the cornea.

36. The system as claimed in claim 27, wherein one of the clinical devices is selected from a group consisting of slit lamps, optical arrangements for 3D measuring and to check geometry in accordance with Scheimpflug, systems for optical coherence tomography (OCT), systems for topography, systems for wave front diagnostics and systems for pachymetry.

37. The system as claimed in claim 27, wherein the patient positioning system supporting the patient is guided on a track and the patient positioning system is movable to position the patient
in a first position wherein the patient's eye is located in a first treatment area of one of the clinical devices to create a flap cut in the surface of the cornea,
in a second position wherein the patient's eye is located in a second treatment area of one of the clinical devices to ablate the tissue of the cornea, or
in a third or further position wherein the patient's eye is located in a measuring or monitoring area of a clinical device to diagnose the eye, and wherein the notification tools comprise systems for position detection and/or the stopping of the patient positioning system.

38. The system as claimed in claim 37, wherein the patient positioning system:
is located on a circular track, where the first, second, third and further positions are positions on this track, or
takes place by rotating the patient positioning system around a rotating axis (24) which is situated between the middle and the foot end of the patient positioning system (3), or
takes place by moving the patient along a straight track, where the preferred positions are positions on this track.

39. The system as claimed in claim 27, further comprising at least one entry and exit area for the patient.

40. The system as claimed in claim 27, wherein the patient positioner further comprises at a positioning drive;
the positioning drive is operably connected to a positioning control;
the positioning control transmits control commands to the positioning drive, and
the positioning is operable to control and confirm preset positions.

41. The system as claimed in claim 40, wherein the positioning control further comprises
an input module for the manual setting of the control commands, or
a control for the automatic generation of the control commands by means of distance or force values captured by a readings recorder.

42. The system as claimed in claim 41, further comprising an incremental or absolute measuring positioning measurement system or an optical positioning measurement system with a camera module to capture distance values which is operably connected to the positioning system.

43. System as claimed in claim 39, further comprising a positioning control module operably connected to a device for fully automated patient and eye positioning, which comprises a navigation supported drive control having elements to capture and guide the position of the eye and sensors to detect obstacles, wherein the control module is operably connected with one of the sensors and the drive control to facilitate avoiding obstacles.

44. The system as claimed in claim 36, further comprising, to position the patient:
a headrest for the patient and a mobile contact glass that can be placed on the eye and through which the laser beam will penetrate the eye,
a force readings recorder for reading the force that the contact glass is applied with, and
a safety mechanism operably coupled with the force readings recorder that will move the contact glass and the headrest apart if the contact glass is applied to the eye using excessive force.

45. The system as claimed in claim 44, wherein the safety mechanism does not initiate the separation of the contact glass from the headrest until a preset limit value has been exceeded and adjusts the relative position of these two items accordingly if the limit value is not reached.

46. The system as claimed in claim 44, further comprising a common base body to hold the devices and brackets to attach the devices to the base body.

47. The system as claimed in claim 46, wherein the base body further comprises a base with guided tracks for the patient's movement when the patient positioning system's location is changed relative to the clinical devices.

48. The system as claimed in claim 47, where the guided tracks comprise rails integrated in the base.

49. The system as claimed in claim 27, wherein the notification tools comprise connections for wireless or wired transmission of data selected from a group consisting of a peer to peer system, a server-based network, a bus, network, a star network, and a ring network.

50. The system as claimed in claim 49, wherein the transmission of the data is wired and parts of the network are located inside a base body.

51. The system as claimed in claim 27, where the clinical devices are connected to a central power supply to receive power.

52. The system as claimed in claim 51, wherein the central power supply source comprises an emergency backup power unit.
